(19) United States
(12) Patent Application Publication
(10) Pub. No.: US 2025/0124224 A1
(43) Pub. Date: Apr. 17, 2025

(54) ARCHITECTURES OF LARGE LANGUAGE MODELS FOR MEDICAL TREATMENT DECISION SUPPORT

(71) Applicant: Trialmatch.me, Inc. d/b/a/Trialjectory, Clifton, NJ (US)

(72) Inventors: Tzvia Bader, Stamford, CT (US); Guy Gildor, Stamford, CT (US)

(21) Appl. No.: 18/913,578

(22) Filed: Oct. 11, 2024

Related U.S. Application Data
(60) Provisional application No. 63/544,022, filed on Oct. 13, 2023.

(51) Int. Cl. G06F 40/20; G16H 10/60; G16H 50/20

(52) U.S. Cl. CPC G06F 40/20; G16H 10/60; G16H 50/20

(57) ABSTRACT

Large language models in conjunction with natural language processing models for medical treatment decision support are provided. In some embodiments, a method of training a large language model is provided. The method can comprise obtaining a plurality of terms associated with a medical topic. The method can comprise generating, using a natural language processing (NLP) model, a plurality of prompts based on the plurality of terms. The plurality of prompts are configured to train a large language model (LLM). The method can comprise providing each of the plurality of prompts to the LLM, thereby training the LLM on the plurality of terms.

### RELATED APPLICATION

This application claims the benefit of U.S. Provisional Application. No. 63/544,022 filed Oct. 13, 2023, which is hereby incorporated by reference in its entirety.

### BACKGROUND

Embodiments of the present disclosure relate to systems and methods for medical treatment decision support, and more specifically, to predicting, optimizing, and providing medical treatment options to patients using artificial intelligence (AI) models.

### BRIEF SUMMARY

According to embodiments of the present disclosure, methods of and computer program products for providing and optimizing predictive medical treatment are provided. As described herein, the process can be called PMM (Precision Medical Modeling).

In some embodiments, a method of training a large language model is provided. The method can comprise obtaining a plurality of terms associated with a medical topic. The method can comprise generating, using a natural language processing (NLP) model, a plurality of prompts based on the plurality of terms. The plurality of prompts are configured to train a large language model (LLM). The method can comprise providing each of the plurality of prompts to the LLM, thereby training the LLM on the plurality of terms.

In some embodiments, a method of training a large language model is provided. The method can comprise obtaining a plurality of terms and unstructured text associated with a medical topic. The method can comprise generating, using a natural language processing (NLP) model, a plurality of prompts based on the plurality of terms and the unstructured text. The plurality of prompts are configured to train a large language model (LLM). The method can comprise providing each of the plurality of prompts to the LLM, thereby training the LLM on the plurality of terms and the unstructured text.

In some embodiments, the plurality of terms include a plurality of terms from a given domain.

In some embodiments, the plurality of prompts include a definition of at least one of the plurality of terms.

In some embodiments, the method further comprises providing, to the LLM, a medical record of an individual.

In some embodiments, obtaining the plurality of terms associated with the medical topic further comprises receiving the plurality of terms from a database over a network.

In some embodiments, the method further comprises pre-processing the unstructured text.

In some embodiments, the plurality of prompts includes the plurality of terms as structured text.

In some embodiments, a method of providing medical treatment decision support is provided. The method can comprise receiving a user query. The method can comprise providing the user query to a large language model (LLM). The LLM can be trained based on a plurality of terms provided by an NLP model. The method can comprise receiving from the LLM an unstructured response to the user query. The method can comprise processing the unstructured response using an NLP model to conform the unstructured response with a structured data set. The method can comprise outputting the conformed response to a user.

In some embodiments, the user query includes a request to provide at least one treatment steps for an individual with a condition.

In some embodiments, the unstructured response includes information regarding treatment of a condition.

In some embodiments, the structured data set includes at least one definition of one of the plurality of terms.

In some embodiments, the unstructured response includes one of the plurality of terms, and conforming the unstructured response includes applying the at least one definition to one of the plurality of terms.

In some embodiments, the method further comprises pre-processing the user query with the NLP model.

In some embodiments, the plurality of terms includes structured text.

In some embodiments, the method further comprises providing the user query to one or more NLP model. In some embodiments, the method further comprises receiving from the NLP model a classical response to the user query. In some embodiments, the method further comprises comparing the conformed response with the classical response, thereby detecting hallucination by the large language model.

In some embodiments, the large language model is trained by obtaining a plurality of terms and unstructured text associated with a medical topic, generating, using a natural language processing (NLP) model, a plurality of prompts based on the plurality of terms and the unstructured text, and providing each of the plurality of prompts to the LLM, thereby training the LLM on the plurality of terms and the unstructured text.

A method of providing medical treatment decision support is provided, where a user query is received. The user query is provided to a large language model (LLM). An unstructured response to the user query is received from the large language model. The unstructured response is processed using an NLP model to conform the unstructured response with a structured data set. The conformed response is output to a user.

### BRIEF DESCRIPTION OF THE SEVERAL VIEWS OF THE DRAWINGS

FIG. 1 depicts an exemplary method of training large language models according to embodiments of the present disclosure.

FIG. 2 depicts an exemplary architecture for both training and using large language models in medical treatment decision support according to embodiments of the present disclosure.

FIG. 3 depicts an exemplary architecture for training large language models in medical treatment decision support according to embodiments of the present disclosure.

FIG. 4 depicts an exemplary architecture for using trained large language models in medical treatment decision support according to embodiments of the present disclosure.

FIG. 5 depicts an exemplary application of architectures for training and using large language models in medical treatment decision support according to embodiments of the present disclosure.

FIG. 6 depicts a computing node according to an embodiment of the present disclosure.

### DETAILED DESCRIPTION

Large language models (LLMs) and natural language processing (NLP) are branches of artificial intelligence focused on understanding and processing human language. In general, LLMs excel in generating human-like text, while NLP encompasses a wide range of language processing tasks and offers greater control and specificity for domain-specific applications. LLMs, exemplified by models like GPT-4.5 or Bard, are known for their capabilities in generating coherent and contextually relevant text, answer questions, and even engage in human-like conversations. NLP involves the development of algorithms and models that can analyze, interpret, and manipulate human language such as text classification, sentiment analysis, named entity recognition, machine translation, and information extraction.

For specialized fields such as healthcare and clinical trials, large datasets may be required for ensuring both medical understanding and complex contextual analysis. However, challenges arise in limited datasets that may be obtained from patients that limit the ability to train the LLM for this specific subject.

Embodiments of the present disclosure address these challenges by integrating LLMs with NLP techniques, so as to provide deep medical understanding and efficient complex contextual analysis, resulting in improved accuracy, structure, and complexity. More specifically, embodiments of the present disclosure implement an augmented architecture that integrates an LLM in conjunction with a non-LLM model (e.g., a classical NLP model) on the same text, so as to capitalize on their relative advantages. This process can be referred to as PMM, or Precision Medical Modeling. For example, rather than training the LLM from scratch, various embodiments use either prompt engineering or custom models on the LLM using the outputs of existing NLP models as “suggested answers”. Furthermore, classical NLP models are provided to analyze responses from AI models for verifying answer credibility. Additionally, existing models can be used to determining accuracy of the AI models.

Advantageously, embodiments of the present disclosure leverage advanced AI technology to match patients to tailored treatments, predict their healthcare journey, optimize care and treatment options, identify untapped treatment opportunities, and/or unlock new possibilities in patient care. Accordingly, healthcare providers can make informed decisions that lead to better patient outcomes and improved healthcare efficiency.

A method of training a large language model is provided, where a plurality of terms and unstructured text associated with a medical topic is obtained from a database. Such a database a database containing general medical knowledge, or a database having a particular medical topic (e.g., cancer). The classical NLP can preprocess the unstructured text. The classical NLP model can then generate, based on the plurality of terms, a plurality of training prompts based on the unstructured text and associated terms of the plurality of terms to provide to the LLM for training. The NLP can include information (e.g., hints) in the training prompts that guide the LLM in structuring the information. In some embodiments, after training, the NLP model can provide a plurality of prompts to the LLM based on a user input, causing the LLM to issue a response to those prompts. In some embodiments, the prompts can include information (e.g., hints) to help the LLM structure the text.

In some embodiments, the trained LLM can be updated by changing the LLM's version/provider in response to the results of evaluating the LLM. Changing the LLM's version/provider can be done as part of a version release.

A method of providing medical treatment decision support is provided using the trained LLM as described above. The LLM receives a user query. In some embodiments of the present disclosure, the user query can comprise providing medical documents or a search query for medical treatments. The LLM can output the unstructured response/result to the user query. In some embodiments of the present disclosure, the unstructured response may comprise one or more LLM-generated sentences that include extracted values/criteria and/or additional contexts/explanation by the LLM. The unstructured response is processed using an NLP model to conform the unstructured response with a structured data set. In some embodiments of the present disclosure, the structured data set can comprise a list of criteria (e.g., clinical criteria, biomarkers, symptoms, test results, etc.) for a patient to be eligible for a certain medical treatment. The NLP outputs the conformed response to a user. In some embodiments of the present disclosure, the conformed response can comprise one or more of a list of relevant medical treatments that match a patient's condition, and/or structural medical attributes.

In some embodiments, the user query is provided to one or more NLP model. A classical response to the user query can be received from the NLP model. The conformed response can be compared with the classical response. The comparison can thereby detect hallucination by the large language model if that comparison determines there are no topic-specific terms in the conformed LLM results.

In some embodiments, the large language model is trained according to the method as described herein.

In general, embodiments of the present disclosure include analysis of patient data and/or generation of treatment advice using generative AI models (e.g., an LLM). In some embodiments, the patient data can include medical profiles, biomarker mappings, and/or patent preferences. In some embodiments of the present disclosure, the patient data is incorporated to the user query so that the LLM can extract medical attributes from that patient data or to match to treatment options for that patient. The patient data (e.g., in the form of structured or unstructured text) can also be used during LLM training to verify the accuracy of the LLM output. In some embodiments, the treatment advice generated by the LLM can be structured based on eligibility criteria, novel medical taxonomy, and/or patient matching.

Embodiments of the present disclosure can include steps as further described below.

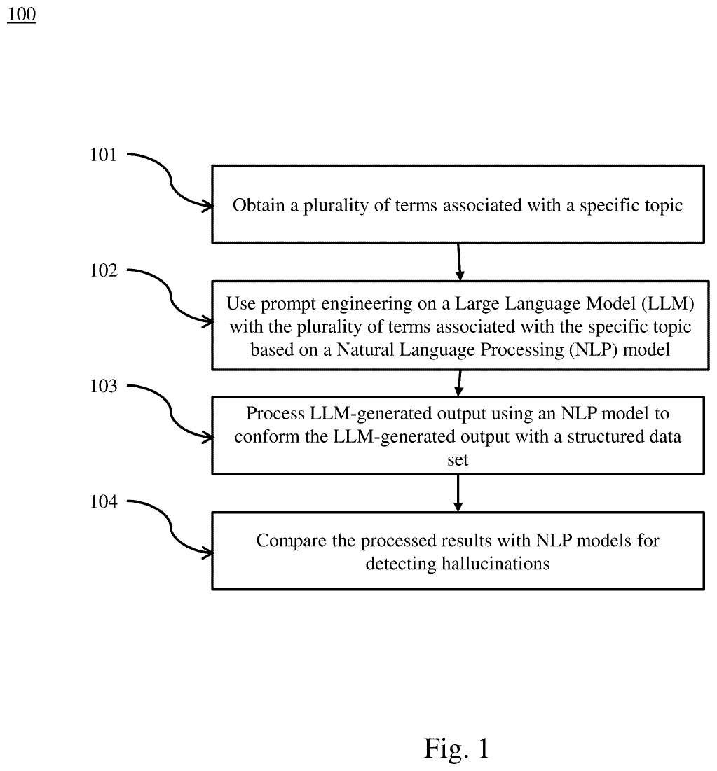

FIG. 1 is a flowchart 100 illustrating an exemplary method of training large language models according to embodiments of the present disclosure. A plurality of terms associated with a specific topic is obtained (101). Prompt engineering is used on a Large Language Model (LLM), with the plurality of terms associated with the specific topic based on a Natural Language Processing (NLP) model (102). The LLM-generated output is processed using an NLP model to conform the LLM-generated output with a structured data set (103). The processed results are compared with NLP models for detecting hallucinations (104).

At step 101, a plurality of terms associated with a medical topic is obtained for training a Large Language Model (LLM). In some embodiments, the plurality of terms can include cancer type, disease type, disease status, disease stage, and/or treatment type.

In some embodiments, the LLM does not incorporate some or all of the plurality of terms directly. In such embodiments, an NLP generates prompts to train the LLM on missing terms. In some embodiments, a set of regular expressions (regex) and synonyms derived from an existing data dictionary are provided to the LLM. For example, in the context of cancer biomarkers, the LLM can be trained to recognize the term EGFR as a synonym for other terms such as NISBD2, ERBB, ERBB1, ERBB2. An exemplary LLM prompt configured to train the LLM can be “The cancer biomarker EGFR can sometimes be referred to as NISBD2, ERBB, ERBB1, erb-b2 or PIG61,” however a person of ordinary skill in the art can recognize the prompt can take other forms.

In some embodiments, the plurality of terms are provided from a library of terms. One or more NLP models can then process those plurality of terms, identify and categorize items from a corpus, and generate a list of synonyms for those terms. For example, the one or more NLP models can identify and categorize terms from a relevant corpus of data (such as medical literature), and generate a list of synonyms for the terms. In some embodiments, the plurality of terms is associated with a topic (e.g., cancer). A database (e.g., a corpus of data) associated with the topic, and a structural database can be provided to one or more NLP models to extract terms associated with the topic. In some embodiments, the plurality of terms may include terms that appear in clinical trials inclusion or exclusion criteria. For example, the plurality of terms can be extracted from a database (e.g., clinicaltrials.gov) in an automated way, or based on manual tagging. In some embodiments of the present disclosure, the topic of interest, along with a relevant corpus of data (such as the clinicaltrials.gov database) is provided to one or more NLP models that extract topic-associated terms from the corpus of data.

In some embodiments, a question relating to a specific topic is provided to the LLM for training. In some embodiments, the question may comprise a question about a medical condition or an individual's medical records.

At step 102, a Large Language Model (LLM) is trained with the plurality of terms associated with the medical topic based on the results of the Natural Language Processing (NLP) model. After training, the LLM can generate high quality output results.

In some embodiments, the LLM model is trained by providing unstructured text through the LLM associated with pre-quantified questions. In some embodiments, the unstructured text is preprocessed by one or more NLP models before being provided to the LLM. In some embodiments, the pre-quantified questions include reduced number of words within a close set of terms, such as “choose one of the following answers . . . ”. The pre-quantified questions can be used as another way of training and the improving accuracy and precision of the LLM. The pre-quantified questions match the unstructured text to specific terms/words, thereby improving model efficacy. The pre-quantified questions can also be used to simplify the user query.

In some embodiments, the unstructured text includes clinical trial description and the patient medical records.

In some embodiments, the NLP models generate prompts using Hidden Markov models. In some embodiments, the NLP models include a trained Bidirectional Encoder Representations from Transformers (BERT) model. In some embodiments, the NLP models include one or more clustering models.

At step 103, LLM-generated results can be processed using a NLP model (e.g., a dedicated NLP model) to conform the LLM-generated results with a structured data set. In some embodiments, the output from the LLM includes a reference to a source text or a location within the source text on which the LLM-generated results are based. Accordingly, the NLP processes the LLM-generated results by verifying the text in such location in the source text, followed by feeding the text of the LLM-generated results to an NLP model.

At step 104, the results obtained at step 103 are compared with the output of one or more NLP models to detect hallucinations.

In some embodiments, the comparison between the LLM output and NLP output is based on whether topic-specific terms (e.g., terms relevant to the topic of the question used for training the LLM) are included in the output (e.g., text provided by LLM-generated results for feeding to the NLP model). In some embodiments, a question can be provided for training the LLM. The question can comprise matching approved drugs to specific medical conditions or biomarkers found via next-generation sequencing. In some cases, if no topic-specific terms are included in the output (e.g., topic-specific terms are not detected in the output), the LLM output is considered to include a hallucination. In some cases, if topic-specific terms are included in the output, the LLM output is determined to be correct even if the NLP disagrees with the LLM on the meaning of the term (e.g., one model considers the question as an inclusion criteria and the other model considers the question as an exclusion criteria).

In some embodiments, if a hallucination is detected, the LLM is rerun with caveats. In some embodiments, the caveats can be determined via NLP or logic through a quality assurance process. For example, a caveats can comprise a rule to not match treatments for a recurrent patient or for a newly diagnosed patient. If a hallucination is detected again, the source documents that the output is based on can be tagged with NULL values as for the specific question/query or attribute.

In some embodiments, the method as described herein further includes a text preprocessing step including word embedding (e.g., word2vec, GloVe) and/or bag-of-words (e.g., TFIDF) for preprocessing. In some embodiments, the NLP-generated prompts are preprocessed before being provided to the LLM for model training.

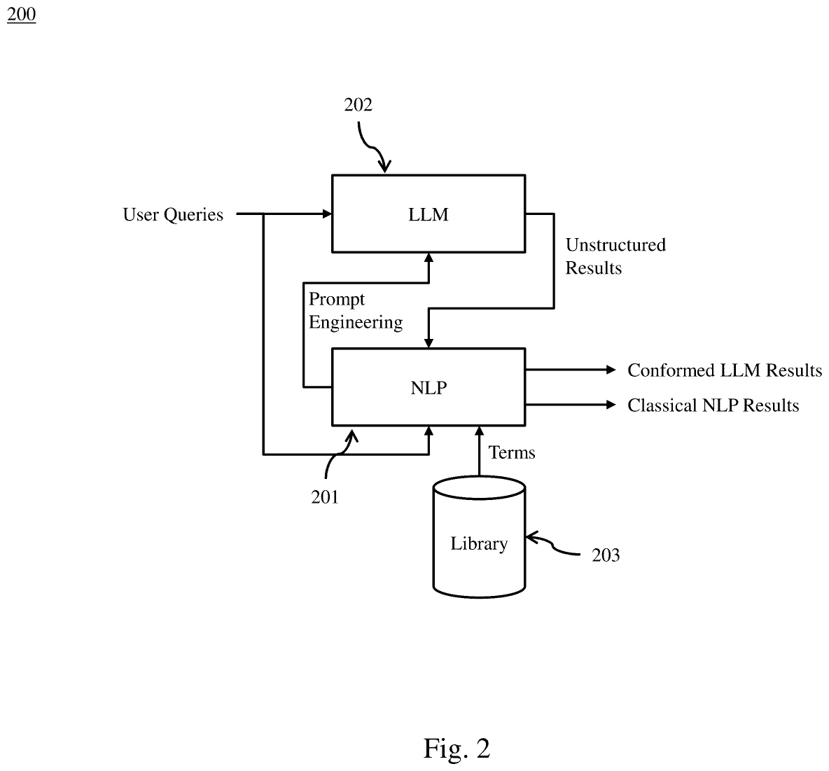

FIG. 2 is a diagram 200 illustrating an exemplary architecture of an NLP model to train an LLM and for the NLP to confirm results from the LLM according to embodiments of the present disclosure. NLP component(s) 201 is used to generate engineered prompts to LLM 202 in order to train LLM in a given medical domain as set forth above. In some embodiments, the engineered prompts are generated based on terms drawn from library 203. A user query is provided to both the NLP model 201 and LLM 202. The LLM 202 generates unstructured results in response to the user query. The unstructured result is conformed to a predetermined structure by NLP component(s) 201. In some embodiments, NLP component(s) 201 also generates a classical NLP result to a user query. This classical NLP result may be compared to the conformed LLM result in order to detect disparities that may indicate a hallucination by the LLM.

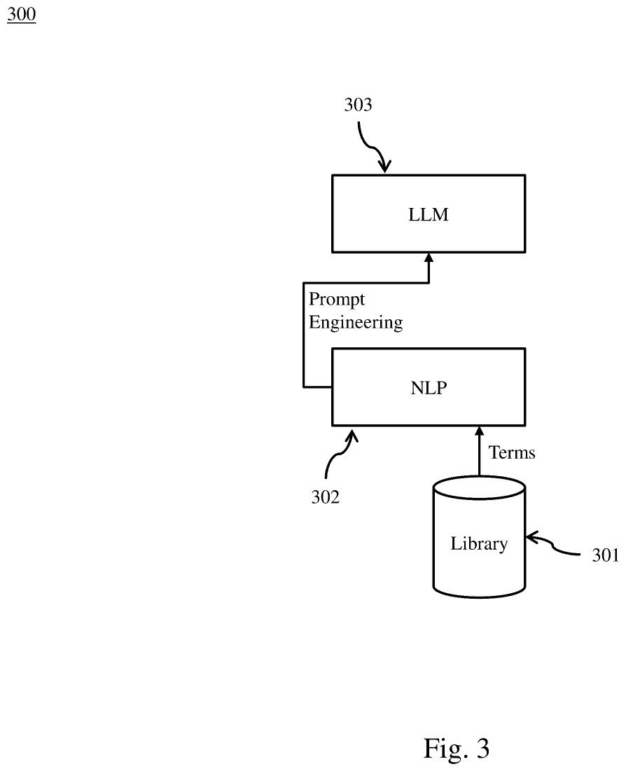

FIG. 3 is a diagram 300 illustrating an exemplary architecture for training a LLM 303 in medical treatment decision support according to embodiments of the present disclosure. NLP model(s) 302 are used to generate engineered prompts to an LLM 303 to train the LLM in a given medical domain as set forth above. In some embodiments of the present disclosure, the engineered prompts are generated based on terms drawn from a library 301. In some embodiments, the engineered prompts can include definitions or synonyms of terms in the given medical domain.

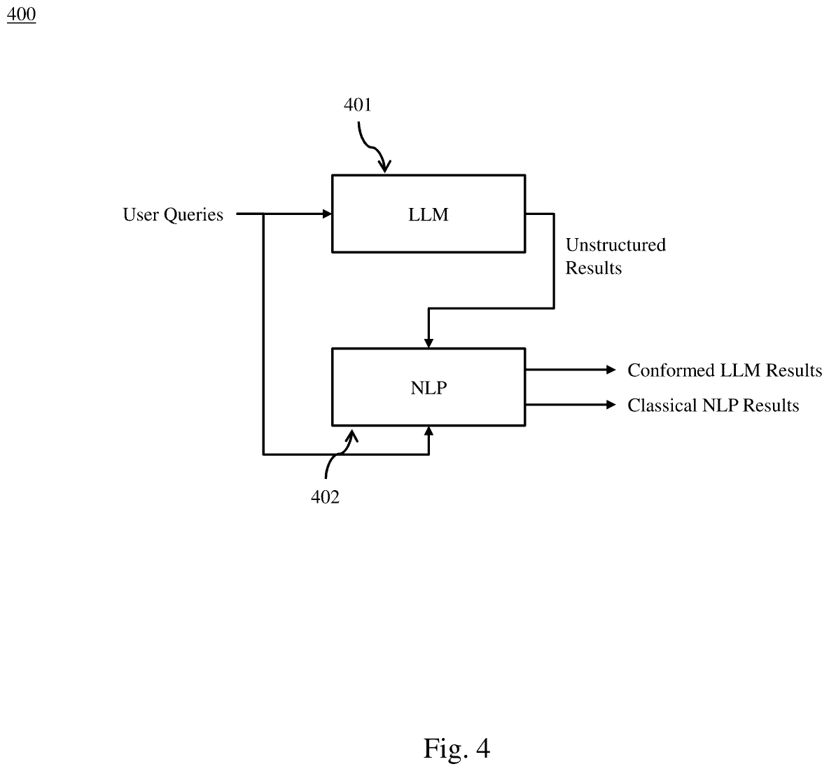

FIG. 4 is a diagram 400 illustrating an exemplary architecture for using a large language model 401 in medical treatment decision support according to embodiments of the present disclosure. In some embodiments, the large language model 401 can be the trained large language model 303 illustrated by FIG. 3. In relation to FIG. 4, a user query is provided to both the NLP model(s) 402 and the LLM 401. The user query can specify the medical domain of interest. The LLM 401 generates unstructured results in response to the user query. The unstructured result is conformed to a predetermined structure by NLP models(s) 402. In some embodiments, NLP models(s) 402 also generates a classical NLP result to a user query. This classical NLP result may be compared to the conformed LLM result to detect disparities that may indicate a hallucination by the LLM.

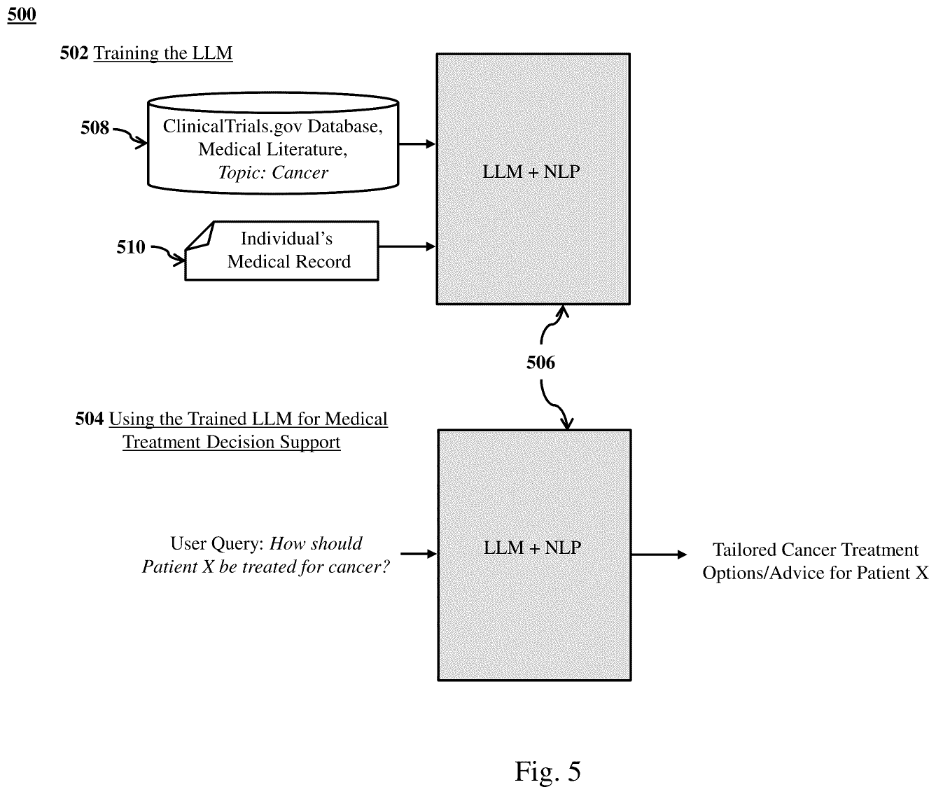

FIG. 5 is a diagram 500 illustrating an exemplary application of architectures for training and using large language models in medical treatment decision support according to embodiments of the present disclosure. To train LLM +NLP system 506, a corpus 508 of general medical information and a topic of interest are provided to an NLP of the system 506. The corpus 508 can be provided to the system over a network, such as the Internet. The system 506 can also an individual patient's medical record 510. (502). To use the trained system for medical treatment decision support for the patient, a user query is provided (e.g., from a medical provider or from the patient) to the system, and the system outputs tailored treatment options/advice for the patient (504).

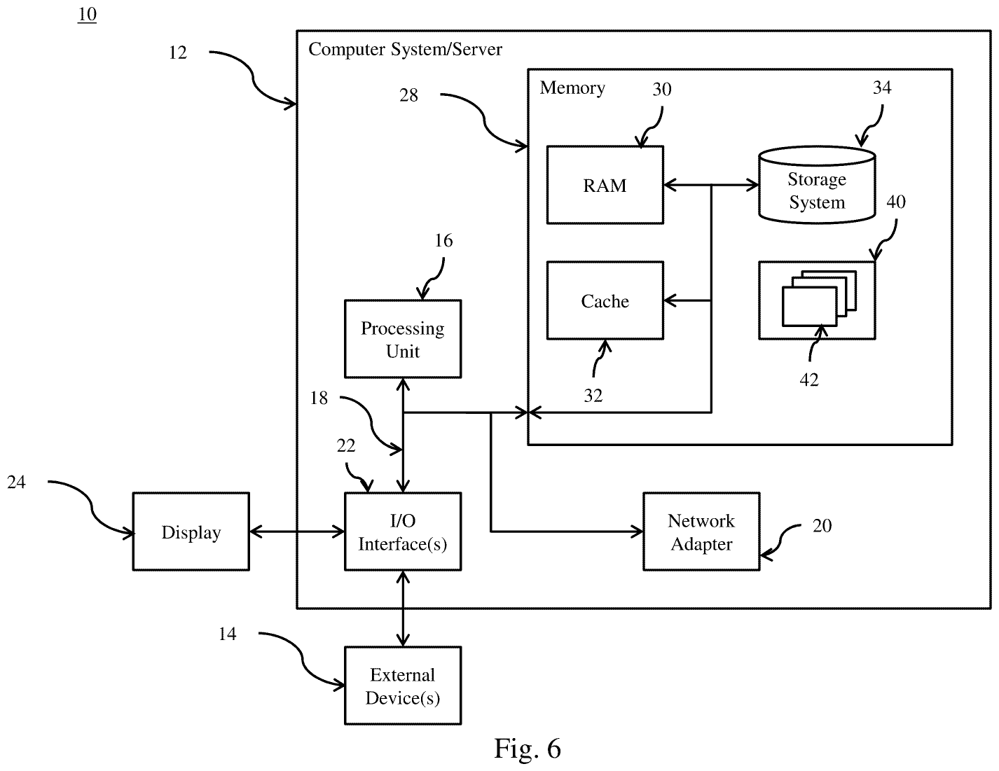

Referring now to FIG. 6, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 6, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method of training a large language model, the method comprising:
obtaining a plurality of terms and unstructured text associated with a medical topic;
generating, using a natural language processing (NLP) model, a plurality of prompts based on the plurality of terms and the unstructured text, the plurality of prompts being configured to train a large language model (LLM); and
providing each of the plurality of prompts to the LLM, thereby training the LLM on the plurality of terms and the unstructured text.

2. The method of claim 1, wherein the plurality of terms include a plurality of terms from a given domain.

3. The method of claim 1, wherein the plurality of prompts include a definition of at least one of the plurality of terms.

4. The method of claim 1, further comprising: providing, to the LLM, a medical record of an individual.

5. The method of claim 1, wherein obtaining the plurality of terms associated with the medical topic further comprises receiving the plurality of terms from a database over a network.

6. The method of claim 1, further comprising pre-processing the unstructured text.

7. The method of claim 1, wherein the plurality of prompts includes the plurality of terms as structured text.

8. A method of providing medical treatment decision support, the method comprising:
receiving a user query;
providing the user query to a large language model (LLM), the LLM trained based on a plurality of terms provided by an NLP model;
receiving from the LLM an unstructured response to the user query;
processing the unstructured response using an NLP model to conform the unstructured response with a structured data set; and
outputting the conformed response to a user.

9. The method of claim 8, wherein the user query includes a request to provide at least one treatment steps for an individual with a condition.

10. The method of claim 8, wherein the unstructured response includes information regarding treatment of a condition.

11. The method of claim 8, wherein the structured data set includes at least one definition of one of the plurality of terms.

12. The method of claim 11, wherein the unstructured response includes one of the plurality of terms, and conforming the unstructured response includes applying the at least one definition to one of the plurality of terms.

13. The method of claim 8, further comprising pre-processing the user query with the NLP model.

14. The method of claim 8, wherein the plurality of terms includes structured text.

15. The method of claim 7, further comprising:
providing the user query to one or more NLP model;
receiving from the NLP model a classical response to the user query; and
comparing the conformed response with the classical response, thereby detecting hallucination by the large language model.

16. The method of claim 7, wherein the large language model is trained by:
obtaining a plurality of terms and unstructured text associated with a medical topic;
generating, using a natural language processing (NLP) model, a plurality of prompts based on the plurality of terms and the unstructured text; and
providing each of the plurality of prompts to the LLM, thereby training the LLM on the plurality of terms and the unstructured text.
